(19) United States
(12) Patent Application Publication
(10) Pub. No.: US 2024/0389933 A1
(43) Pub. Date: Nov. 28, 2024

(54) BRUXISM, SLEEP, AND DENTAL HEALTH MONITORING PLATFORM

(71) Applicants: Rich ABLE, Kirkland, WA (US); Joseph N. KENNELLY ULLMAN, Seattle, WA (US)

(72) Inventors: Rich ABLE, Kirkland, WA (US); Joseph N. KENNELLY ULLMAN, Seattle, WA (US)

(21) Appl. No.: 18/693,318

(22) PCT Filed: Sep. 21, 2022
(86) PCT No.: PCT/US2022/044266

§ 371 Date: Mar. 19, 2024

Related U.S. Application Data
(60) Provisional application No. 63/246,491, filed on Sep. 21, 2021.
(60) Provisional application No. 63/313,197, filed on Feb. 23, 2022.

(51) Int. Cl. A61B 5/00; A47C 31/00; A47G 9/10; A61B 5/0205; A61B 5/11; A61B 5/1455; G05B 19/042

(52) U.S. Cl. CPC A61B 5/4557; A61B 5/0022; A61B 5/0205; A61B 5/1118; A61B 5/14551; A61B 5/4818; A61B 5/682; A61B 5/746; A61B 5/747; G05B 19/042; A47C 31/008; A47G 9/10; A61B 2560/0223; A61B 2562/0219; A61B 2562/0252; A61B 2562/164; G05B 2219/2614

(57) ABSTRACT

A health monitoring device, platform and related methods for assessing, tracking, analyzing, diagnosing and/or treating bruxism, sleep disorders and/or other dental health conditions or disorders. In some instances, a health monitoring device is provided in the form of a wearable mouthpiece having a plurality of sensors configured to track impact and/or force data over time associated with one or more bruxing events (e.g., teeth grinding and/or clenching), thereby allowing a healthcare professional to monitor the severity and frequency of grinding and/or clenching over time.

### BACKGROUND

### Technical Field

This disclosure relates to a health monitoring device, platform and related methods for assessing, tracking, analyzing, diagnosing and/or treating bruxism, sleep disorders and/or other dental health conditions or disorders.

### Description of the Related Art

The nocturnal grinding of the teeth is a common condition for large segments of the population. This condition presents itself in pediatric dentistry patients and extends through senior care. There are two main types of bruxism: one occurs during sleep (nocturnal bruxism) and one during wakefulness (awake bruxism). Dental damage may be similar in both types, but the symptoms of sleep bruxism tend to be worse on waking and improve during the course of the day. The symptoms of awake bruxism may not be present at all on waking, and then worsen over the day. The causes of bruxism are not completely understood, but probably involve multiple factors. Awake bruxism is more common in women, whereas men and women are affected in equal proportions by sleep bruxism. Awake bruxism is thought to have different causes from sleep bruxism. Several treatments are in use, although there is little evidence of robust efficacy for any particular treatment.

Presently, there is no ability to assess, track, or analyze the impact of teeth grinding or nocturnal bruxism.

### BRIEF SUMMARY

Embodiments of the present invention are directed to a bruxing monitoring device that includes an instrumented mouthpiece configured to be worn by an individual before they retire for the night and throughout their sleep cycle. The mouthpiece is configured to track impact and/or force data over time associated with one or more bruxing events (e.g., teeth grinding and/or clenching), thereby allowing a healthcare professional to monitor the severity and frequency of grinding and/or clenching over time. Further investigation by the healthcare professional may facilitate treatment of the cause of the symptom at an earlier time than might have been otherwise possible. Related systems and methods are also provided. Embodiments also include a monitoring device, platform and related methods for assessing, tracking, analyzing, diagnosing and/or treating other dental health conditions or disorders and/or sleep disorders, such as, for example, sleep apnea. In addition, it is appreciated that embodiments may be utilized by an individual throughout the day, including periods when the individual is not asleep.

### BRIEF DESCRIPTION OF THE SEVERAL VIEWS OF THE DRAWINGS

FIG. 1 is a perspective view of one example embodiment of a health monitoring device in the form of a wearable mouthpiece.

FIG. 2 is a top plan view of the health monitoring device of FIG. 1 showing example locations of a plurality of sensor devices for capturing grinding and/or clenching data.

FIG. 3 is a top plan view of a health monitoring device showing example locations of optional acoustic sensors.

FIG. 4 is a top plan view of a health monitoring device showing example locations of optional temperature sensors.

FIG. 5 is a diagram of a sensing module suitable for use in the health monitoring devices and systems disclosed herein.

FIG. 6 is a system diagram of a health monitoring system, according to one example embodiment.

FIG. 7 is a system diagram of a health monitoring system, according to another example embodiment.

FIG. 8 is a perspective view of one example embodiment of a health monitoring device in the form of a flexible strip with embedded sensor devices.

FIG. 9A is a perspective view of one example embodiment of a health monitoring device with a removable sensor board in an unassembled configuration.

FIG. 9B is a perspective view of the health monitoring device of FIG. 9A in an assembled configuration.

FIG. 10 is a system diagram of a health monitoring system, according to one example embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with wearable health monitoring devices have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

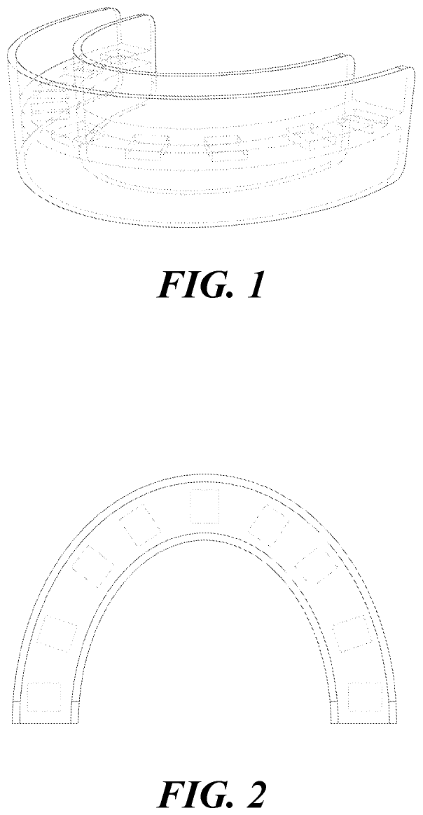

FIG. 1 shows one example embodiment of a wearable health monitoring device in the form of a mouthpiece to be worn by an individual. The mouthpiece includes a plurality of sensors (e.g., accelerometers, pressure transducers and/or strain gauges) positioned adjacent one another within portions of the mouthpiece that allow for secure fit and coverage of the user's teeth. For example, the mouthpiece may include two or more strain gauges (or other sensors) in communication with electrical components for collecting bruxing event data during at least a portion of one or more bruxing events. The strain gauges may collect data related to forces generated during bruxing events over time in one or more targeted areas of the user's dental arches.

As discussed further below, the mouthpiece is created in a manner that allows the device to monitor the impact of the clenching and/or grinding of the upper and lower jaw and subsequent accelerations and/or forces over time exerted on the teeth during one or more bruxing events.

With reference to FIG. 2, the plurality of sensors (e.g., accelerometers, pressure transducers and/or strain gauges) may be placed in a pattern so as to capture acceleration and/or force data from all areas of the clenching zone—that is from a substantial entirety of the plan view of the user's dental arches, including on both sides of the dental midline.

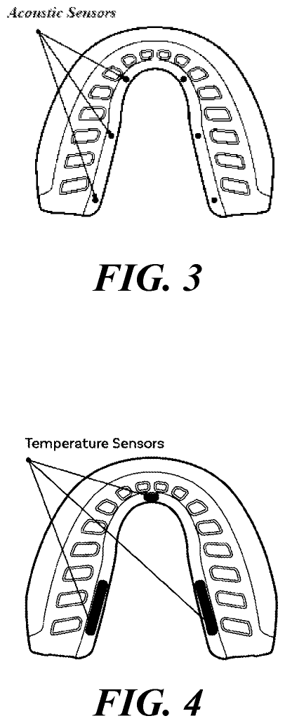

With reference to FIG. 3, the mouthpiece may also contain one or more acoustic monitoring sensors to assess sleep apnea and related events such as momentary cessation of breathing and interrupted airflow during sleep. These acoustic monitoring sensors will deliver meaningful data for the dentist or other healthcare professional to determine the degree of severity and onset of this condition. The acoustic monitoring sensors may be communicatively coupled to local memory or external memory for selectively storing acoustic data for further processing and/or may be communicatively coupled to a wireless communication module for selectively transmitting acoustic data for further processing.

With reference to FIG. 4, the mouthpiece may also contain one or more temperature sensors to collect temperature data of the user's mouth. These temperature sensors may be used to assess patient health and/or to provide other functionality, such as detecting when the mouthpiece is in position for use. The temperature sensors may be communicatively coupled to local memory or external memory for selectively storing temperature data for further processing and/or may be communicatively coupled to a wireless communication module for selectively transmitting temperature data for further processing.

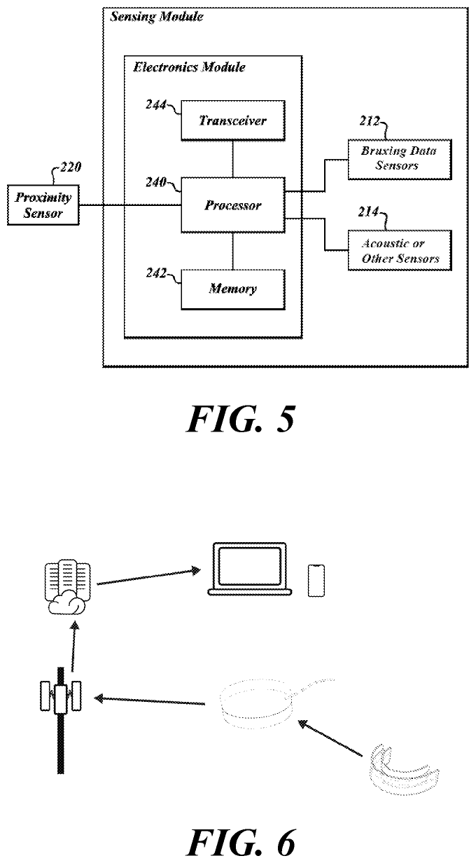

The health monitoring device may include onboard components to collect, store, and transmit data from the various aforementioned sensors for evaluation later or in real time. For example, with reference to FIG. 5, the health monitoring device may include a processor 240 and a memory 242 in communication with the processor 240. The memory 242 may include stored programming instructions operable by the processor 240 to allow the processor 240 to collect data from the plurality of bruxing data sensors 212 and optionally acoustic or other sensors 214 and to store such data in the memory 242. In some embodiments, one or more additional sensors, such as a proximity sensor 220, may be provided to determine when the health monitoring device is in place and worn by the user. The proximity sensor 220 or other such sensor is likewise in signal communication with the processor 240 and is preferably associated with the strain gauge or other bruxing data sensors 212 to confirm that the sensor data is valid because the health monitoring device (e.g., mouthpiece) is in use. A transceiver 244 is also provided in signal communication with the processor 240 for transmitting the bruxing data or other data to one or more external computing devices or networks. In addition, an onboard power supply (e.g., battery) is provided to supply power to the electronics. The power supply may be wirelessly chargeable through a base charging device or the like.

It should be appreciated that while the aforementioned components (e.g., processor, memory) are described as being provided by a mouthpiece positioned in the mouth, in some embodiments, some components may be positioned in an accompanying device (e.g., charging base or case) that acts a charging station for the mouthpiece and optionally provides other functionality. For example, the accompanying device may include processors and other sensors such as acoustic sensors, light sensors, proximity sensors, temperature sensors, or other sensors. The accompanying device may be positioned near the user's sleeping area so as to collect data while the user sleeps, to assess the quality of the user's sleep. The accompanying device may collect and transmit data to other local computers (e.g., desktop computer, smartphone) or to computers or servers over a network, such as a wireless communications network.

In some embodiments, the health monitoring device is configured to allow data gathered by the health monitoring device to be transferred to a remote computer for subsequent evaluation. Thus, in one configuration as illustrated in FIG. 6, a wireless transmitter (such as 5G cellular) is provided allowing the data to be wirelessly transferred to a host computer, server or network that can store the sensor data. In some instances, the host computer may further include stored programming instructions allowing for the collection and evaluation of the sensor data. In some versions, the host computer may be configured to store the collected data over time, further calculating average data (such as average grind or most impactful event data at particular locations in the mouth), thereby allowing the computer to compare data for a particular day or time with a baseline average value.

The host computer may likewise be in communication with a remote computer, directly or over a network such as the Internet. In some instances, the remote computer may be that of a dentist or other healthcare professional. The remote computer may be configured to evaluate the data in the same fashion as identified above, or may further include similar data aggregated from a multitude of patients in order to allow comparison of individual data or trends from a single user with aggregated data or trends from a larger population. The remote computer may also be a tablet or mobile phone.

In some embodiments, the charging case may be connected to a local computer that may collect and store the data similarly to the host computer described previously. The local computer may send the data to the host computer or other devices such as a mobile phone or tablet via Wi-Fi, Bluetooth, or similar wireless communication technology.

In yet other embodiments, the sensor data may be transmitted directly from the processor in the mouthpiece to a local device such as a computer, tablet, or mobile phone over Bluetooth, Wi-Fi, or similar.

In some instances, the charging case, local computer, remote computer, or all devices may include threshold values stored in memory and programming instructions that cause an alarm to be triggered if certain criteria are met.

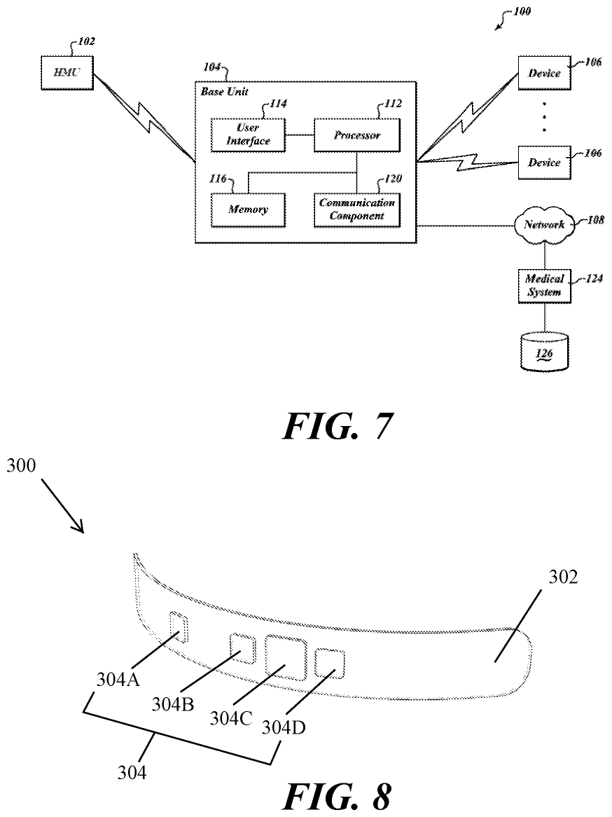

FIG. 7 illustrates an exemplary system 100 that performs collection of bruxing information received from at least one health monitoring unit (HMU) 102 and makes the bruxing information available to relevant parties. The health monitoring unit 102 is a mouthpiece as described above that incorporates one or more sensors that are configured to collect impact and/or force data over time associated with grinding and/or clenching of teeth during at least a portion of one or more bruxing events. The system 100 includes a base unit 104 (e.g., charging base or communications base) that is in wireless communication with the health monitoring unit 102 and is optionally in wired or wireless communication with one or more devices 106. The health monitoring unit 102 may be directly coupled to the base unit 104, or may alternatively pass its data to the base unit 104 indirectly, through a server, network, or other electronic device. The base unit 104 includes a processor 112, a user interface 114, local memory 116, and a communication component 120. The base unit 104 may receive bruxing information wirelessly from the health monitoring unit 102 and optionally makes that data available to the one or more additional devices 106.

In some versions, the base unit 104 or any of the devices 106 are in wired or wireless connection with a medical system 124 over a public or private data network 108. The medical system 124 receives bruxing and other information from the base unit 104 or the devices 106 for analysis with regard to stored patient information and/or storage into a database 126.

According to some embodiments, the health monitoring device may also include a proximity or position sensor, as previously discussed with reference to FIG. 5. The proximity or position sensor can be configured to determine whether the mouthpiece is in place and being worn at the prescribed times for compliance measurement. In one example, the position sensor may be an optical or electrical resistance sensor to detect moisture (saliva) at the location of the sensor. The sensor is directed inward, to detect moisture (saliva) received on the mouthpiece. In such a case, the processor would conclude that the mouthpiece is being worn by the user. Other forms of sensors may also be used, such as temperature sensors (See FIG. 4), piezoelectric sensors, capacitive sensors, magnetic sensors or other devices. In each case, the proximity or position sensor is in signal communication with the processor so that the memory can store data from the proximity or position sensor.

In addition to determining compliance (that the user is properly wearing the mouthpiece) the proximity or position sensor, if configured to measure saliva, could be used to assess conditions related to dry mouth. The position sensor, especially if an optical sensor, may be able to monitor conditions related to tooth and gum health including plaque build-up and gingivitis. The same sensor may be able to detect the color of the teeth to monitor staining as it relates to the desire for tooth whitening.

FIG. 8 is a perspective view of one example embodiment of a health monitoring device 300 with embedded sensor devices. The device 300 includes a body 302 in the form of a flexible, thin strip of material. The material of the body 302 may be a selected material, such as the material typically used in the dental care industry for whitening strips. The dimensions of the body 302 may also be selected to correspond to a user's teeth. In some embodiments, the body 302 includes an adhesive on a surface of the body 302 facing the teeth for adhering the body 302 to the teeth. Further, the body 302 may have a width (i.e., from top to bottom in the orientation shown in FIG. 8) that is adapted to be received only on one surface of the user's teeth, such as an outward facing surface or an inward facing surface of the teeth, or the body 302 may have a width such that the body 302 is folded over to contact both front and rear surfaces of the user's teeth.

The device 300 is designed to capture breathing and force measurement, among other characteristics of a user's activities during sleeping or waking hours. To that end, the device 300 includes a sensor array 304 including a plurality of sensors 304A-304D. In the example embodiment shown in FIG. 8, the sensor array 304 is embedded in the body 302 of the device 300 such that the sensors 304A-304D are permanently affixed to the body 302. In some embodiments, the sensors 304A-30D are completely received within the body 302 (i.e., internal to the body 302). In one non-limiting example, the body 302 may include two layers of material with the sensors 304A-304D coupled to a first layer. A second layer is coupled to the first layer of the body 302 to sandwich the sensors 304A-304D between the layers. Other configurations are also contemplated herein. For example, in at least some embodiments, the sensors 304A-304D may be embedded in the body 302 but at least partially extend from a surface of the body 302, such as from a front or rear surface of the body 302. Still further, the sensors 304A-304D may be coupled to an external surface of the body 302 such that the sensors 304A-304D are not embedded in the body 302.

The sensors 304A-304D may be formed as flexible circuits, including but not limited to integrated circuits disposed on flexible substrates, to enable the body 302 to conform to the shape of the teeth of the user, including folding around the teeth, without damaging the sensors 304A-304D. The sensors 304A-304D may be any sensor of the type described herein. In a preferred embodiment, the sensors 304A-304D include sensors to detect and measure cardiorespiratory variables of the user, including but not limited to respiration sensors and audio sensors that detect respiration spectra (i.e., respiration intensity, duration, common breath sounds, type of breathing such as nasal or through the mouth) during waking and non-waking hours and also while the user is at rest or physically active, or both. Still further, the sensors 304A-304D may include one or more accelerometers to establish movement characteristics of the user during sleeping and waking hours.

The accelerometers may also assist with calibration of the device 300 by establishing baseline posture and relative axes of the posture of the user, including with respect to the jaw or head, neck, and spinal position and architecture of the user. In some embodiments, the calibration process may further include the use of disposable sensor stickers with one or more accelerometers. The stickers are temporarily attached to the body 302 for a calibration process and then can be removed before normal use of the device 300. Alternatively, the stickers may remain in place after calibration until disposal of the device 300. The accelerometers help the device 300 measure whether the user is standing up, laying down, laying on their side, or laying on their stomach as well as to measure the user's walking characteristics to establish gait dynamics. This data from the accelerometers can be interpreted in view of the other data collected by the sensor array 304 to determine how a user's movement characteristics impact bruxing events and respiration spectra, among other benefits.

The sensors 304A-304D may also include fiber optic sensors in a preferred embodiment. The fiber optic sensors measure airflow dynamics, such as air flow rate, volume, velocity, and other air flow characteristics. Still further, the sensors 304A-304D may include pulse oximeter sensors or pulp inflammation marks, or both, that measure the percentage of oxygen saturation and pulp inflammation to determine whether the user is experiencing sleep apnea or other sleep issues. Further, the sensors 304A-304D may include position sensors and moisture sensors to determine whether the user is wearing the device 300 and to measure the user's saliva to detect possible dry mouth issues, as described herein.

In some embodiments, the sensor array 304 further includes any of the hardware components described herein, such as the components described with respect to FIG. 5. Thus, the device 300 may store the data collected by the sensor array 304 in a memory and transmit the data to an external device for further review and processing, such as by a doctor, dentist, or other medical professional. The sensor array 304 may include hardware, such as various receivers, transmitters, or transceivers to support communication of the data from the sensor array 304 to the external device according to any communication protocol now known or developed in the future. The sensor array 304 may also include an electronics module of the type described with reference to FIG. 5 for communication between the device 300 and other devices, as described elsewhere herein.

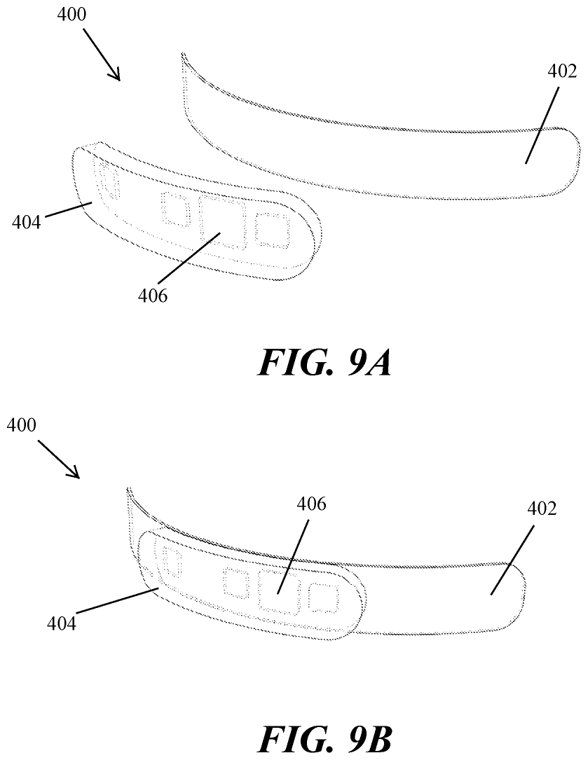

The device 300 in FIG. 8 is designed to be disposable, in some embodiments. However, the present disclosure contemplates different configurations to avoid disposal of the sensors to lower costs for the user. For example, in FIG. 9A and FIG. 9B, a health monitoring device 400 includes a body 402 and a sensor array 404 removably coupled to the body 402. In FIG. 9A, the body 402 and the sensor array 404 are in an unassembled configuration with the sensor array 404 uncoupled from the body 402 while in FIG. 9B, the body 402 and the sensor array 404 are in an assembled configuration with the sensor array 404 coupled to the body 402.

With continuing reference to FIG. 9A and FIG. 9B, the body 402 of the device 400 may be a mouth guard or a flexible strip of the type described herein, along with any other device designed to be worn on a user's teeth or in a user's mouth. The sensor array 404 may include sensors 406 shown in dashed lines that are any of the sensors described herein. The sensors 406 may be embedded in the sensor array 404 or may be coupled to an external surface of the sensor array 404. Further, the sensor array 404 may be removably coupled to the body 402 with an adhesive, such as a glue or tape, in some embodiments. Alternatively, the sensor array 404 may be magnetically coupled to the body 402 in addition to any other removable fastening method. In some embodiments, the sensor array 404 is a flexible sticker with an adhesive on a surface facing the body 402 such that the user can attached the sensor array 404 to the body 402. The device 400 may further include a removable strip on the adhesive for packaging and transport to the user with the user removing the cover strip before application of the sensor array 404 to the body 402.

In operation, the user attaches the sensor array 404 to the body 402 with an acceptable fastening method and then attaches the body 402 to their teeth. The sensors 406 of the sensor array 404 then perform the functions described herein to provide information regarding various characteristics of the user during waking or sleeping hours, including movement characteristics, cardiorespiratory spectra (i.e., breathing intensity, duration, sounds, and type of breathing, among others), baseline posture and relative position of the user, airflow dynamics, oxygen saturation, pulp inflammation, whether the user is wearing the device, and other characteristics. The device 400 and the sensor array 404 may enable measurement of the above and other characteristics during a selected period of time, such as only during waking hours, only during sleeping hours, or during both sleeping and waking hours, along with a selected time period (i.e., between certain hours of the day or for a selected length of time in hours), with the length of time of use and the type of use (i.e., during sleeping or waking hours) recommended by a medical professional. The selected time period may also include periods of physical activity as well as a resting condition of the user.

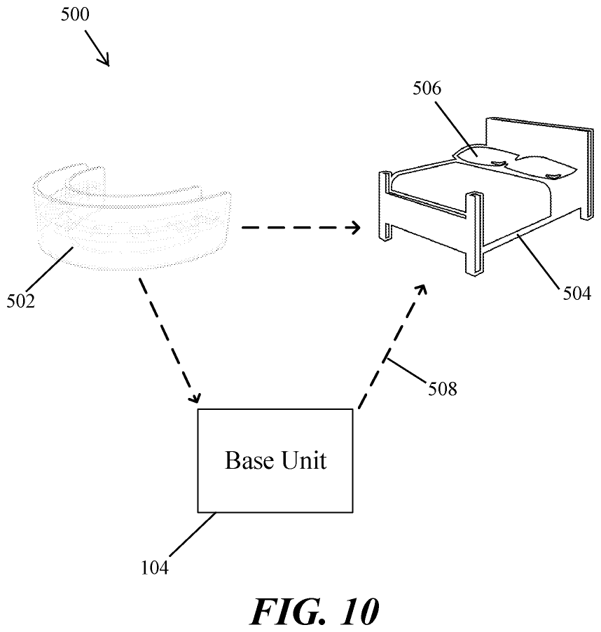

FIG. 10 is a system diagram of a health monitoring system 500 according to one or more embodiments of the disclosure. The system 500 includes a mouthpiece 502, a bed 504, a pillow 506, and the base unit 104. The mouthpiece 502 may be any of the mouthpieces described herein, including the mouth guard type mouthpiece in FIGS. 1-4, as well as the flexible strip health monitoring devices 300, 400 in FIGS. 8-9B. The bed 504 and the pillow 506 are illustrated schematically in FIG. 10 and may be any one of a number of commercially available beds and pillows, or may be specially adapted to enable the concepts of the disclosure. In some embodiments, the bed 504 includes at least one characteristic that is adjustable in response to an input from the user, such as at least one of an elevation, a temperature, an amount of support, and a firmness of the bed 504 in some non-limiting examples. As used above, the term “elevation” refers to a bed with a mattress that inclines or declines relative to a generally horizontal base in order to change a position of at least the upper body of the user on the bed relative to the generally horizontal base. Thus, the bed 504 is also configured to incline or decline or otherwise adjust the user's sleeping position in some embodiments.

It is to be appreciated that the bed 504 may include an embedded or onboard controller carried by the bed 504 or an external controller in communication with the bed 504 wherein the controller is configured to receive an input from the user and adjust the at least one characteristic of the bed in response to the input. The controller may include at least one memory for storing instructions and at least one processor for executing the instructions stored in the memory to adjust the at least one characteristic. Further, the bed 504 may include sensors in communication with the controller of the bed 504 for detecting sleeping parameters of the user and automatically adjusting the at least one characteristic accordingly.

As generally indicated in FIG. 10 with dashed arrows 508, the mouthpiece 502, the bed 504, the pillow 506, and the base unit 104 may be in communication with each other. For example, the mouthpiece 502 may communicate with the base unit 104 and the base unit 104 may communicate with the bed 504 or the controller of the bed 504. Alternatively, the mouthpiece 502 may communicate directly with the bed 504 or the controller of the bed 504 and the base unit 104 may be omitted in some embodiments. Such communication may occur wirelessly according to any known communication protocol and through the use of receivers, transmitters, or transceivers in the mouthpiece 502, the base unit 104, the bed 504, and/or the pillow 506. The base unit 104 may also have a wired connected to the bed 504 in some embodiments.

In operation, the sensors of the mouthpiece 502 collect data of the type described herein during a selected period of time, which may be a non-waking or a sleeping period of the user while on the bed 504. The data collected by the sensors of the mouthpiece 502 may generally correspond to a number of different sleep quality characteristics. In other words, the data collected by the sensors on the mouthpiece 502 may indicate that the user is not experiencing quality sleep, or that certain issues, such as bruxing, can be improved by adjusting the at least one characteristic of the bed 504. In some non-limiting examples, the sensors on the mouthpiece 502 may detect that the user is experiencing a bruxing event and transmit a signal to the base unit 104 corresponding to the detected bruxing event. The base unit 104 may then determine an appropriate adjustment to characteristics of the bed 504 and transmit a signal or instructions to the bed 504 or the controller 504 of the bed 504 to adjust the at least one characteristic of the bed 504, such as elevation or the firmness of the bed 504, or both, accordingly. The determination of the appropriate adjustment to the bed 504 may be stored in the memory of the base unit 104 or in the memory of the controller of the bed 504 where the base unit 104 is omitted. The same process may be used if the mouthpiece 502 detects snoring or any other condition described herein.

In an embodiment, in addition to or in lieu of the bed 504, one or more pillows 506 may be provided that have one or more characteristics that are adjustable, such as a temperature, an amount of support, and a firmness of the pillow 506, among other characteristics. Thus, in addition to or in lieu of adjusting the at least one characteristic of the bed 504, the base unit 104 may transmit instructions to adjust the one or more characteristics of the one or more pillows 506. Each pillow 506 may have an embedded or onboard controller for receiving the instructions directly from the base unit 104 and adjusting the at least one characteristic of the pillow 506. In an embodiment wherein both a bed 504 and a pillow 506 are provided, the pillow 506 may be in direct communication with the bed 504 rather than the base unit 104 such that the instructions are communicated from the base unit 104 to the bed 504, and then from the bed 504 to the pillow 506. Such communication may occur wirelessly according to any known communication protocol and through the use of receivers, transmitters, or transceivers in the mouthpiece 502, the base unit 104, the bed 504, and/or the pillow 506. The base unit 104 may also have a wired connected to the pillow 506 in some embodiments.

In some embodiments, the adjustment of the at least one characteristic of the bed 504 and/or the pillow 506 is an iterative process. For example, if the mouthpiece 502 detects snoring, the mouthpiece 502 and the base unit 104 may instruct the bed 504 to adjust the elevation of the bed 504 or a firmness of the pillow 506, or both, for a selected period of time, such as one to five minutes or more or less. If the mouthpiece 502 continues to detect the condition after the selected period of time, the mouthpiece and the base unit 104 may instruct the bed 504 to further adjust the elevation or instruct the pillow 506 to further adjust the firmness, or any of the other characteristics of the bed 504 and/or the pillow 506 until the condition detected by the mouthpiece 502 is below a selected threshold value (i.e., a volume below a certain decibel level for snoring or a force impact below a certain selected force level for bruxing, etc.). In this way, the mouthpiece 502 may also be used in conjunction with the base unit 104, the bed 504, and/or the pillow 506 to improve the quality of the user's sleep and reduce the occurrence of certain sleep defects.

In at least one embodiment, the system 500 further stores data corresponding to the conditions detected by the mouthpiece 502 and the corresponding adjustments of the characteristics of the bed 504 and/or the pillow 506 as well as the impact of the adjustments of the bed 504 and/or pillow 506 to the conditions detected by the mouthpiece 502 over time. The base unit 104 may then transmit this information over any known protocol to a licensed medical professional for analysis. In one non-limiting example, if the mouthpiece 502 detects snoring and instructs the bed 504 and/or pillow 506 to adjust the at least one characteristic one or more times, as above, but the adjustments do not correct the snoring, then the licensed medical professional can view the data and suggest a treatment plan accordingly. Thus, the system 500 may further enable treatment of certain health issues that may occur during sleep, as detected by the mouthpiece 502.

Further embodiments of the disclosure may incorporate aspects described and shown at least with reference to FIG. 6, FIG. 7, and FIG. 10 to enable direct device to device communication or direct device to network to device communication without the base unit 104. In an embodiment, a mouthpiece and/or sensors carried by the mouthpiece of the type described herein are in communication with one or more other devices directly, and/or with the one or more other devices via at least one intermediary network in a broader Internet of things (“IoT”) system. Such “other” devices may include, but are not limited to, smart home hubs, light sources, televisions, HVAC systems, speakers, mobile devices (such as smartphones, tablets, and others), and the like. Except as otherwise provided below, each device in such an IoT system may include hardware to enable wireless communication between the devices and/or networks in the IoT system, such as at least with respect to the processor 112, memory 116, and communication component 120 of FIG. 7. The wireless communication can be accomplished via a number of known communication protocols, such as WiFi, Bluetooth, cellular networks or Machine to Machine (“M2M”) communication, radio frequency (such as ZigBee or ZWave, among others), and others.

In at least some embodiments, the mouthpieces described herein are in communication with an IoT hub and/or with other IoT devices directly over one of the communication protocols described herein (such as is shown with devices 106 in FIG. 7) or indirectly, such as via network 108 shown in FIG. 7. As a result, the information gathered by sensors carried by the mouthpieces can be used to change the characteristics of multiple devices that are part of the IoT system. For example, if the mouthpiece detects that a user is having difficult falling asleep, the mouthpiece may communicate with a speaker with instructions to play music, to one or more connected light sources to dim (i.e., change an amount of light output by the light sources) or turn on/off the light sources, and/or to a television to turn on/off the television. The instructions may also include instructions for changing a volume output by the speaker and/or television, as well as the content provided to the user via the speaker and/or television, such as preferred songs, channels, or movies, as well as more general genres of categories of content. Similar actions may executed (i.e., play music, turn on one or more light sources, turn on a television to a selected channel) when the mouthpiece detects that the user is awake following a sleep event of a minimum threshold duration that can be selected by a user and may be one hour, four hours, eight hours or more or less in some non-limiting examples. The mouthpieces may also communicate directly with a mobile device, such as a smartphone or tablet, to call an ambulance or alert other emergency services if the mouthpiece detects a lack of breathing, heartbeat, or other characteristics of the user suggesting that a life-threatening event may have occurred. In yet further non-limiting examples, the mouthpieces described herein can be worn by those who experience sleep apnea, with the mouthpiece generally being capable of communicating with a mobile device of a user (which may be the mobile device of the wearer of the mouthpiece or the mobile device of a designated third party) to provide an alarm or alert, such as an audible, visual, and/or haptic signal, if the mouthpiece detects that the user has stopped breathing for a period of time that exceeds a minimum threshold that can be selected by the user or a medical professional (i.e., at least 30 seconds, at least 60 seconds, at least 90 seconds, or more). Such alert can also be provided via a smartwatch or other IoT-enabled wearable device, among others.

As described above, the mouthpiece may also enable communication with connected devices at a medical professional's office, as well as with a bed, pillow, and other aspects to provide the benefits and advantages herein. In yet further non-limiting examples, the mouthpiece may be in direct or indirect communication with an HVAC system to adjust a temperature of a room or space based on the information gathered by the sensors, such as to increase or decrease a temperature in a room if a user is having difficulty falling or staying asleep. The mouthpiece may also communicate with other medical devices of the user, such as a continuous positive airway pressure (CPAP) machine to adjust one or more operational characteristics of the machine based on the information gathered by the sensors on the mouthpiece. For example, if a user is having difficulty breathing, operation of the CPAP machine may be activated, or if already active, settings may be adjusted to improve the user's breathing. Many other configurations are possible and contemplated herein, even if not expressly mentioned. In summary, the mouthpieces and/or sensors carried by the mouthpieces may be in communication with any selected device now known or developed in the future that includes connectivity capability, such as IoT, WiFi, Bluetooth, Radio Frequency, and cellular communication enabled devices in some non-limiting examples.

It will be appreciated that computing systems and devices disclosed herein are merely illustrative and are not intended to limit the scope of embodiments of the present invention. The systems and/or devices may instead each include multiple interacting computing systems or devices, and may be connected to other devices that are not specifically illustrated, including via Bluetooth communication or other direct communication, through one or more networks such as the Internet, via the Web, or via one or more private networks (e.g., mobile communication networks, etc.). More generally, a device or other computing system may comprise any combination of hardware that may interact and perform the described types of functionality, optionally when programmed or otherwise configured with particular software instructions and/or data structures, including without limitation desktop or other computers (e.g., tablets, slates, etc.), database servers, network storage devices and other network devices, smart phones and other cell phones, consumer electronics, wearable devices, biometric monitoring devices, Internet appliances, and various other consumer products that include appropriate communication capabilities. In addition, the functionality provided by the systems disclosed herein may in some embodiments be distributed in various modules. Similarly, in some embodiments, some of the disclosed functionality may not be provided and/or other additional functionality may be available. In addition, in certain implementations various functionality of the system may be provided by third-party partners of an operator of the system. For example, data collected by the system may be provided to a third party for analysis and/or metric generation.

It will also be appreciated that, while various items are described as being stored in memory or on storage while being used, these items or portions of them may be transferred between memory and other storage devices for purposes of memory management and data integrity. Alternatively, in other embodiments some or all of the software modules and/or systems may execute in memory on another device and communicate with the illustrated computing systems via inter-computer communication. Thus, in some embodiments, some or all of the described techniques may be performed by hardware means that include one or more processors and/or memory and/or storage when configured by one or more software programs and/or data structures, such as by execution of software instructions of the one or more software programs and/or by storage of such software instructions and/or data structures. Furthermore, in some embodiments, some or all of the systems and/or modules may be implemented or provided in other manners, such as by consisting of one or more means that are implemented at least partially in firmware and/or hardware (e.g., rather than as a means implemented in whole or in part by software instructions that configure a particular CPU or other processor), including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), etc. Some or all of the modules, systems and data structures may also be stored (e.g., as software instructions or structured data) on a non-transitory computer-readable storage mediums, such as a hard disk or flash drive or other non-volatile storage device, volatile or non-volatile memory (e.g., RAM or flash RAM), a network storage device, or a portable media article (e.g., a DVD disk, a CD disk, an optical disk, a flash memory device, etc.) to be read by an appropriate drive or via an appropriate connection. The systems, modules and data structures may also in some embodiments be transmitted via generated data signals (e.g., as part of a carrier wave or other analog or digital propagated signal) on a variety of computer-readable transmission mediums, including wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of the present disclosure may be practiced with other computer system configurations.

Moreover, aspects and features of the various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

U.S. Provisional Patent Application No. 63/246,491, filed Sep. 21, 2021 and U.S. Provisional Patent Application No. 63/313,197, filed Feb. 23, 2022 are incorporated herein by reference, in their entirety.

1. A health monitoring device, comprising:
a mouthpiece; and
a plurality of sensors carried by the mouthpiece and configured to collect impact and/or force data over time associated with grinding and/or clenching of teeth during at least a portion of one or more bruxing events.

2. The health monitoring device of claim 1, wherein the plurality of sensors are arranged to collect the impact and/or force data from one or more areas associated with a majority of an area of a plan view of the user's dental arches, including opposing sides of the dental midline.

3. The health monitoring device of claim 1, wherein the plurality of sensors are arranged to collect the impact and/or force data from one or more areas associated with a substantial entirety of an area of a plan view of the user's dental arches, including opposing sides of the dental midline.

4. The health monitoring device of claim 1, wherein the plurality of sensors comprise a plurality of strain gauges to measure forces over time associated with the grinding and/or clenching of teeth during the at least a portion of the one or more bruxing events.

5. The health monitoring device of claim 1, wherein the plurality of sensors comprise a plurality of accelerometers to measure acceleration data over time associated with the grinding and/or clenching of teeth during the at least a portion of the one or more bruxing events.

6. The health monitoring device of claim 1, further comprising a processor and a memory integrated into the mouthpiece, the processor being in signal communication with the plurality of sensors.

7. The health monitoring device of claim 1 wherein each of the plurality of sensors are located in the mouthpiece to collect data associated with accelerations and/or forces between teeth of an upper dental arch of a user and teeth of a lower dental arch of the user.

8. The health monitoring device of claim 1 wherein each of the plurality of sensors are selected or calibrated to detect accelerations and/or forces between teeth of an upper dental arch of a user and teeth of a lower dental arch of the user.

9. The health monitoring device of claim 1 wherein the device is configured to monitor and record and/or transmit a history of teeth grinding and/or clenching data over time, the history of teeth grinding and/or clenching data including magnitude and frequency data.

10. The health monitoring device of claim 1, further comprising an acoustic monitoring sensor to collect data associated with one or more acoustic events during a sleeping event in which the device is worn by a user.

11. The health monitoring device of claim 1, further comprising a proximity or position sensor that is configured to detect whether the mouthpiece is positioned within the mouth of a user.

12. The health monitoring device of claim 1, further comprising an optical sensor configured to monitor conditions related to tooth and gum condition.

13. A health monitoring system, comprising:
a mouthpiece having a plurality of sensors that are configured to collect impact and/or force data over time associated with grinding and/or clenching of teeth during at least a portion of one or more bruxing events, and a power supply; and
a charging device configured to selectively receive the mouthpiece and provide power to the power supply of the mouthpiece.

14. The health monitoring system of claim 13 wherein the charging device is provided in the form of a case that is configured to insertably receive the mouthpiece.

15. The health monitoring system of claim 13 wherein the charging device further comprises a processor, a memory, and a communication device, the processor being in signal communication with the plurality of sensors of the mouthpiece via the communication device to receive and store the impact and/or force data over time associated with the grinding and/or clenching of teeth during the at least a portion of the one or more bruxing events.

16. The health monitoring system of claim 13 wherein the mouthpiece and the charging device are configured to monitor and record and/or transmit a history of teeth grinding and/or clenching data over time, the history of teeth grinding and/or clenching data including magnitude and frequency data.

17. A method, comprising:
providing a mouthpiece having a plurality of sensors that are configured to collect impact and/or force data over time associated with grinding and/or clenching of teeth during at least a portion of one or more bruxing events; and
collecting the impact and/or force data over time associated with the grinding and/or clenching of teeth during the at least a portion of the one or more bruxing events.

18. The method of claim 17, further comprising:
transmitting the impact and/or force data over time associated with the grinding and/or clenching of teeth during the at least a portion of the one or more bruxing events to one or more computing devices for storage and/or processing.

19. The method of claim 17, further comprising:
prior to collecting the impact and/or force data over time, detecting whether the mouthpiece is positioned within the mouth of a user.

20. A health monitoring device, comprising:
a mouthpiece; and
a plurality of first sensors carried by the mouthpiece and configured to collect at least one of cardiorespiratory variable data, impact, and/or force data over a selected time period and during a selected activity period.

21. The health monitoring device of claim 20 wherein the mouthpiece is a mouth guard and the plurality of first sensors are carried by a sensor board.

22. The health monitoring device of claim 21 wherein the sensor board is embedded in the mouth guard or is removably coupled to the mouth guard.

23. The health monitoring device of claim 20 wherein the mouthpiece is a flexible strip structured to adhere to teeth.

24. The health monitoring device of claim 23 wherein the plurality of first sensors are carried by a sensor board that is embedded in the flexible strip or removably coupled to the flexible strip.

25. The health monitoring device of any of the preceding claims wherein the cardiorespiratory variable data includes respiration spectra including at least one of respiratory intensity, respiratory duration, respiratory sounds, and respiratory breathing type.

26. The health monitoring device of any of the preceding claims wherein the selected time period includes at least one of selected waking hours and selected sleeping hours.

27. The health monitoring device of any of the preceding claims wherein the selected activity period includes at least one a rest activity period and a physically active activity period.

28. The health monitoring device of claim 20 further comprising:
a plurality of second sensors configured to collect baseline posture and relative axes data for calibration of the plurality of first sensors.

29. The health monitoring device of claim 28 wherein the plurality of second sensors are carried by the mouthpiece and/or are carried by disposable stickers and/or by disposable strips.

30. The health monitoring device of any of the preceding claims wherein the plurality of first sensors includes a plurality of fiber optic sensors configured to collect airflow dynamic data and/or a plurality of intraoral pulse oximetry sensors to measure selected markers.

31. The health monitoring device of any of the preceding claims wherein the plurality of first sensors include flex circuits, audio sensors and/or accelerometers to capture data on bruxing, air flow measurement, and auditory characteristics.

32. A method, comprising:
providing a mouthpiece having a plurality of first sensors that are configured to collect at least one of cardiorespiratory variable data, impact, and/or force data over a selected time period and during a selected activity period; and
collecting the cardiorespiratory variable data, impact, and/or force data over time during the selected time period and during the selected activity period.

33. The method of claim 32, further comprising:
transmitting the cardiorespiratory variable data, impact, and/or force data over time during the selected time period and during the selected activity period to one or more computing devices for storage and/or processing.

34. The method of claim 32, further comprising:
prior to collecting the cardiorespiratory variable data, impact, and/or force data over time during the selected time period and during the selected activity period, detecting whether the mouthpiece is positioned within the mouth of a user.

35. The method of claim 32 further comprising:
prior to collecting cardiorespiratory variable data, impact, and/or force data over time during the selected time period and during the selected activity period, establishing baseline posture and relative axes to calibrate the mouthpiece with a plurality of second sensors carried by the mouthpiece, disposable stickers and/or disposable strips.

36. A method according to any of claims 20-31.

37. A health monitoring system, comprising:
a mouthpiece;
a plurality of sensors carried by the mouthpiece and configured to collect data over a selected time period, the data including at least one of cardiorespiratory variable data, impact, and/or force data; and
a bed and/or a pillow in communication with the plurality of sensors and having at least one characteristic, the bed and/or the pillow configured to adjust the at least one characteristic in response to the data.

38. The health monitoring system of claim 37 wherein the at least one characteristic of the bed is at least one of an elevation, a temperature, an amount of support, and a firmness of the bed.

39. The health monitoring system of claim 37 wherein the at least one characteristic of the pillow is at least one of a temperature, an amount of support, and a firmness of the pillow.

40. The health monitoring system of claim 37 further comprising:
a controller in communication with the plurality of sensors and the bed and/or the pillow, wherein during operation, the controller receives the data from the plurality of sensors, determines the at least one characteristic of the bed and/or the pillow, and transmits a signal to the bed and/or the pillow to adjust the at least one characteristic.

41. The health monitoring system of claim 40 wherein the controller is external to the bed and/or the pillow.

42. The health monitoring system of claim 40 wherein the controller is integral with the bed and/or the pillow or carried by the bed and/or the pillow.

43. The health monitoring system of claim 40 wherein the controller is a first controller external to the bed and/or the pillow, the system further comprising:
a second controller integral with the bed and/or the pillow or carried by the bed and/or the pillow, the second controller in communication with the first controller, wherein during operation, the second controller receives the signal from the first controller and executes instructions to adjust the at least one characteristic of the bed and/or the pillow.

44. A method according to any of claims 37-43.

45. A health monitoring system, comprising:
a mouthpiece;
a plurality of sensors carried by the mouthpiece and configured to collect data over a selected time period, the data including at least one of cardiorespiratory variable data, impact, and/or force data; and
an external device in communication with the plurality of sensors and having at least one characteristic, the external device configured to adjust the at least one characteristic in response to the data.

46. The health monitoring system of claim 45, wherein the external device is in communication directly with the mouthpiece.

47. The health monitoring system of claim 45, wherein the external device is in communication indirectly with the mouthpiece via one or more intermediary networks.

48. The health monitoring system of claim 45, wherein the external device is one or more of: a speaker, a television, a light source, a mobile device, a bed, a pillow, an HVAC device, a medical device, a computer, a wearable device, and a smart home hub.

49. The health monitoring system of claim 45, wherein the external device is a speaker and the at least one characteristic of the speaker is at least one of: activation or deactivation of music playback, a volume of music playback, and content for music playback.

50. The health monitoring system of claim 45, wherein the external device is a light source, and the at least one characteristic of the light source is at least one of: activation of the light source, deactivation of the light source, and an amount of light output by the light source.

51. The health monitoring system of claim 45, wherein the external device is an HVAC device, and the least one characteristic of the HVAC device is an output temperature of air from the HVAC device.

52. The health monitoring system of claim 45, wherein the external device is a mobile device, and the least one characteristic of the mobile device is at least one of an alert and activation of a communication to an emergency service provider or a medical provider.

53. The health monitoring system of claim 45, wherein the external device is a television, and the least one characteristic is at least one of: activation of the television, deactivation of the television, a volume output by the television, and content displayed to the user via the television.
